(19) United States
(12) Patent Application Publication
(10) Pub. No.: US 2010/0249571 A1
(43) Pub. Date: Sep. 30, 2010

(54) SURGICAL NAVIGATION SYSTEM WITH WIRELESS MAGNETORESISTANCE TRACKING SENSORS

(75) Inventors: Vernon Thomas Jensen, Draper, UT (US); William Hullinger Huber, Scotia, NY (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/415,039

(22) Filed: Mar. 31, 2009

(51) Int. Cl. A61B 5/05

(52) U.S. Cl. 600/409

(57) ABSTRACT

A surgical navigation system having one or more wireless magnetoresistance sensors, where the sensors have the noise and dynamic range appropriate for position and orientation tracking. The surgical navigation system comprising at least one wireless magnetoresistance reference sensor rigidly attached to at least one anatomical reference of a patient, at least one wireless magnetoresistance sensor attached to at least one device, and at least one processor for determining the position and orientation of the at least one device.

### BACKGROUND OF THE INVENTION

This disclosure relates generally to surgical navigation systems for use in minimally invasive surgeries, and more particularly to a surgical navigation system utilizing wireless magnetoresistance tracking sensors.

Surgical navigation systems track the precise position and orientation of surgical instruments, implants or other medical devices in relation to multidimensional images of a patient's anatomy. Additionally, surgical navigation systems use visualization tools to provide the surgeon with co-registered views of these surgical instruments, implants or other medical devices with the patient's anatomy.

The multidimensional images may be generated either prior to or during the surgical procedure. For example, any suitable medical imaging technique, such as X-ray, computed tomography (CT), magnetic resonance (MR), positron emission tomography (PET), ultrasound, or any other suitable imaging technique, as well as any combinations thereof may be utilized. After registering the multidimensional images to the position and orientation of the patient, or to the position and orientation of an anatomical feature or region of interest, the combination of the multidimensional images with graphical representations of the navigated surgical instruments, implants or other medical devices provides position and orientation information that allows a medical practitioner to manipulate the surgical instruments, implants or other medical devices to desired positions and orientations.

Current surgical navigation systems typically include position and orientation tracking sensors, or sensing sub-systems based on electromagnetic (EM), radio frequency (RF), optical (line-of-sight), and/or mechanical technology. The feasibility of using surgical navigation tracking technology for a broader range of clinical applications typically depends on sensor size, affordability, accuracy and sensing range.

Large form factor sensors like optical sensors require an unobstructed line-of-site between the sensors and camera system. This limitation prevents optical sensors from being located inside the body. Even though the tip position of a rigid instrument or other medical device having these sensors attached thereto may be extrapolated, an optical navigation system is therefore, less accurate and unreliable when considering the opportunities for errors inherent in these systems. Optical sensors may be wireless, but they cannot be made small enough to be located at the tip of an instrument or medical device to be inserted into a patient's body in a minimally invasive manner. In order to track the tip of a flexible medical device or tool, such as a catheter, needle or flexible endoscope, it is necessary to locate the sensor at the distal end, which is inserted into the body. Optical tracking sensors may not be used because they require unrestricted line-of-site pathway between the optical target and the image receptor.

EM sensors are typically implemented with coils or microcoils to generate and detect magnetic fields. While coil based EM sensors have been successfully implemented, they suffer from poor signal-to-noise ratio (SNR) as the transmitter coil frequency is reduced and/or the receiver coil volume is reduced. Reducing the SNR translates into a reduced effective tracking range (distance from transmitter to receiver) of the EM sensors that may result in a clinically meaningful position error.

Another problem typically associated with coil based EM sensors is that they are susceptible to magnetic field distortions that arise from eddy currents in nearby conducting objects. This is particularly evident when operating at higher frequencies. The tracking technique used with coil based EM sensors relies on a stable magnetic field, or a known magnetic field map. Therefore, unpredictable disturbances resulting from metallic objects in the magnetic field reduce the accuracy or may even render the tracking technique useless. Selecting a magnetic field frequency as low as the application allows reduces problems resulting from eddy currents, however it also reduces the sensitivity of coil based EM sensors since these are based on induction.

Other problems associated with coil based EM sensors is that they are difficult and expensive to manufacture and are also inherently sensitive to parasitic inductance and capacitance from the cables, connectors and electronics because the sensor signal is proportionally smaller while the parasitic signal remains the same. In addition, parasitic capacitances and inductances are in phase with the signal. Therefore, it is impossible to distinguish between the actual sign and parasitic signal when using a phase-sensitive measurement. While some of the parasitic contributions may be partially nulled out using more expensive components and manufacturing processes, the remaining parasitic inductance and capacitance result in a reduced range.

In addition to coil based EM sensors, there are a large variety of magnetic sensors with differing price and performance attributes. Hall effect-sensors are typically used to detect fields down to approximately 10⁻⁶ Tesla. These sensors are stable, compact, relatively inexpensive and have a large dynamic range. Anisotropic magnetoresistive (AMR) sensors can detect fields down to approximately 10⁻⁹ Tesla. While these sensors are compact and relatively inexpensive, they are highly prone to drift and have a small dynamic range. Therefore AMR sensors need to be reinitialized frequently using high current pulses. Fluxgate magnetometers can detect fields down to approximately 10⁻¹¹ Tesla. However these sensors are expensive, bulky and have a relatively small dynamic range. SQUID magnetometers can detect fields down to approximately 10⁻¹⁵ Tesla. They are also expensive with significant operating costs since they require cryogens or a high-power closed-cycle cooling system.

Therefore, there is a need for a surgical navigation system that includes wireless magnetoresistance tracking sensors having a small form factor, excellent signal-to-noise ratio, excellent low frequency operation, lower sensitivity to parasitic inductance and capacitance, lower sensitivity to distortion, and are very low cost to manufacture.

### BRIEF DESCRIPTION OF THE INVENTION

In accordance with an aspect of the disclosure, a surgical navigation system comprising at least one wireless magnetoresistance reference sensor rigidly attached to an anatomical reference of a patient, at least one wireless magnetoresistance sensor attached to at least one device, and at least one processor for determining the position and orientation of the at least one device.

In accordance with an aspect of the disclosure, a surgical navigation system comprising at least one wireless magnetoresistance sensor attached to at least one device, a wireless planar sensor array positioned on a table supporting a patient undergoing a medical procedure, and at least one processor for determining the position and orientation of the at least one device.

In accordance with an aspect of the disclosure, an integrated circuit device for use in a surgical navigation system comprising a magnetoresistance sensor, and additional circuitry coupled to the magnetoresistance sensor, wherein the magnetoresistance sensor and the additional circuitry coupled to the magnetoresistance sensor operate wirelessly.

Various other features, aspects, and advantages will be made apparent to those skilled in the art from the accompanying drawings and detailed description thereof.

### BRIEF DESCRIPTION OF THE DRAWINGS

FIG. 1 is an enlarged side view of an exemplary embodiment of a magnetoresistance sensor;

FIG. 2 is a schematic diagram of an exemplary embodiment of a magnetoresistance sensor with additional circuitry integrated within an integrated circuit device;

FIG. 3 is a schematic diagram of an exemplary embodiment of a surgical navigation system;

FIG. 4 is a block diagram of an exemplary embodiment of a surgical navigation system;

FIG. 5 is a schematic diagram of an exemplary embodiment of a surgical navigation system; and

FIG. 6 is a block diagram of an exemplary embodiment of a surgical navigation system.

### DETAILED DESCRIPTION OF THE INVENTION

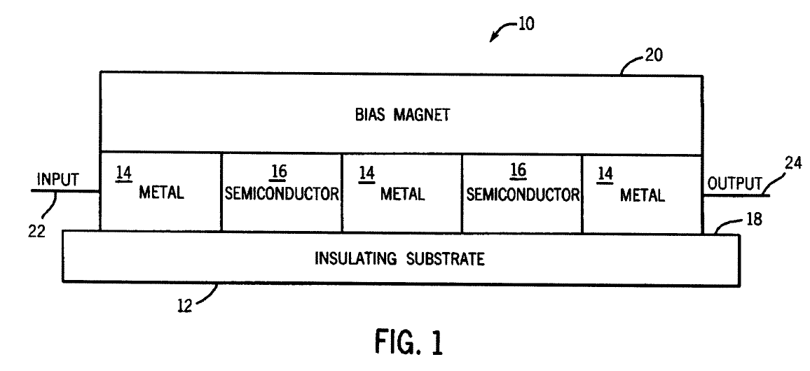

Referring now to the drawings, FIG. 1 illustrates an enlarged side view of an exemplary embodiment of a magnetoresistance sensor 10. A magnetoresistance device is a device that provides a change in electrical resistance of a conductor or semiconductor when a magnetic field is applied. The device's resistance depends upon the magnetic field applied. As shown in FIG. 1, the a magnetoresistance sensor 10 comprises an insulating substrate 12, an alternating pattern of a metal material 14 and a semiconductor material 16 deposited on a surface 18 of the insulating substrate, and a bias magnet material 20 deposited over the alternating pattern of metal material 14 and semiconductor material 16. The alternating pattern of metal material 14 and semiconductor material 16 creates a composite structure with alternating bands of metal material 14 and semiconductor material 16. At least one input connection contact 22 is coupled to the metal material 14 and at least one output connection contact 24 is coupled to the metal material 14.

The semiconductor material 16 may be series connected to increase the magnetoresistance sensor 10 resistance. In an exemplary embodiment, the semiconductor material 16 may be comprised of a single semiconductor element. The bias magnet material 20 subjects the semiconductor material 16 to a magnetic field required to achieve required sensitivity. The magnetoresistance sensor 10 provides a signal in response to the strength and direction of a magnetic field. In an exemplary embodiment, the magnetic field may be approximately 0.1 to 0.2 Tesla.

The application of a magnetic field confines the electrons to the semiconductor material 16, resulting in an increased path length. Increasing the path length, increases the sensitivity of the magnetoresistance sensor 10. The magnetic field also increases the resistance of the magnetoresistance sensor 10. In the geometry disclosed in FIG. 1, at a zero magnetic field, the current density is uniform throughout the magnetoresistance sensor 10. At a high magnetic field, the electrons (or holes) propagate radially outward toward the corners of the semiconductor material 16, resulting in a large magnetoresistance (high resistance).

Many new clinical applications include tracking of a variety of devices including catheters, guidewires, implants, and other endovascular or gastrointestinal instruments that require sensors to be very small in size (millimeter dimensions or smaller). The active area of the magnetoresistance sensor 10 may be scaled to sizes less than 0.1 mm×0.1 mm.

In an exemplary embodiment, the magnetoresistance sensor may be built with various architectures and geometries, including, giant magnetoresistance (GMR) sensors, and extraordinary magnetoresistance (EMR) sensors.

The magnetoresistance sensor 10 provides a very small form factor, excellent signal-to-noise ratio (low noise operation), and excellent low frequency response. Low noise combined with wide dynamic range enables the magnetoresistance sensor 10 to be used for position and orientation tracking in surgical navigation systems. The low frequency response of the magnetoresistance sensor 10 allows a surgical navigation system to operate at very low frequencies where metal tolerance is maximized. In addition, the magnetoresistance sensor 10 is very inexpensive to manufacture, provides improved accuracy and increased sensing range.

The magnetoresistance sensor 10 has very low power requirements that allow for wireless utilization. In an exemplary embodiment, the magnetoresistance sensor 10 may be wireless, with the sensor 10 being driven by self-contained circuitry, data acquisition being performed by self-contained circuitry, and power being provided by a self-contained power source. In an exemplary embodiment, the magnetoresistance sensor 10 may include power conversion and drive circuitry for energizing the sensor. By way of example, a drive current may be supplied by the drive circuitry to energize the sensor, and thereby generate a magnetic field that is detected by another sensor acting as a receiver. In an exemplary embodiment, the magnetoresistance sensor 10 may be powered by a small battery or through inductive coupling.

Wireless operation of the magnetoresistance sensor 10 allows for the development of complete self-contained medical systems or subsystems that may be fabricated onto high-density integrated circuit devices with very low power requirements. This facilitates integration into medical instruments and devices in such a manner as not to negatively influence ergonomic design considerations and for tracking inside a body.

In the context of a surgical navigation system, the magnetoresistance sensor 10 may be attached to or integrated into a surgical instrument (e.g., an imaging catheter, a diagnostic catheter, a therapeutic catheter, a guidewire, a debrider, an aspirator, a handle, a guide, etc.), a surgical implant (e.g., an artificial disk, a bone screw, a shunt, a pedicle screw, a plate, an intramedullary rod, etc.), or some other device. Depending on the clinical application, any number of medical devices may be used. In an exemplary embodiment, there may be more than one device, and more than one magnetoresistance sensor 10 attached to or integrated into each device. In an exemplary embodiment, the magnetoresistance sensor 10 may be attached to the tissue, a bone or an organ of a patient.

As an example for use of the magnetoresistance sensor 10 in an existing clinical application, the magnetoresistance sensor 10 may be integrated within a self-contained gastrointestinal imaging device formed within a capsule for diagnosing and treating gastrointestinal diseases and ailments. A patient swallows the capsule, and the device operates while the capsule progresses by means of natural body function, through the digestive tract. A small battery may be integrated into the design of the device for providing power. Image data is correlated with position data, and is both collected and stored internal to the device, and also transmitted via wireless communication circuitry and protocols as needed. Bi-directional wireless communication circuitry and protocols are used to access data, and for control of programmable parameters of the device.

In an exemplary embodiment, the at least one magnetoresistance sensor 10 may be a battery-powered wireless transmitter or a passive transmitter. In an exemplary embodiment, the at least one magnetoresistance sensor 10 may be a battery-powered wireless receiver or a passive receiver.

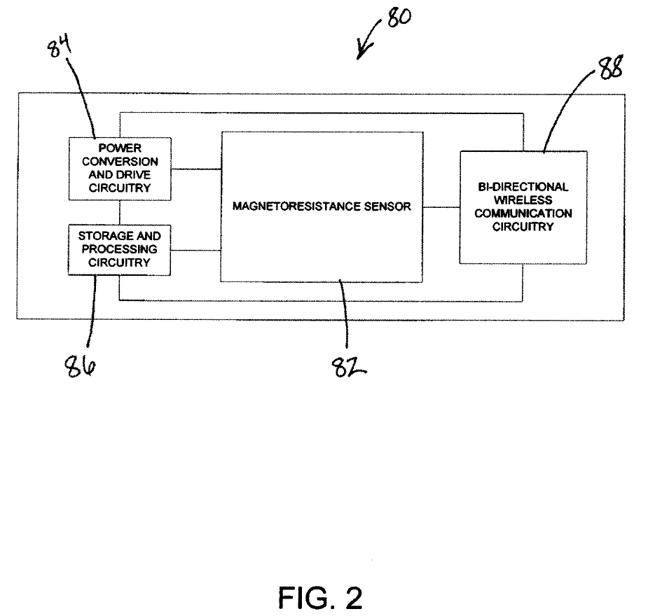

FIG. 2 illustrates a schematic diagram of an exemplary embodiment of a magnetoresistance sensor 82 with additional circuitry 84, 86, 88 integrated within an integrated circuit device 80. In an exemplary embodiment, the integrated circuit device 80 with the magnetoresistance sensor 82 produced thereon may include additional circuitry 84, 86, 88 for different configurations and/or applications. For example, the integrated circuit device 80 may include power conversion and drive circuitry 84 coupled to the magnetoresistance sensor 80 for converting power from a battery or through inductive coupling to a drive current or voltage for energizing the magnetoresistance sensor 80, and also providing power to the additional circuitry 86, 88. As another example, the integrated circuit device 80 may include storage and processing circuitry 86 coupled to the magnetoresistance sensor 82 for storing and processing data from and to the magnetoresistance sensor 82, and also storing and processing data for the additional circuitry 84, 88. And as another example, the integrated circuit device 80 may include bi-directional wireless communication circuitry and protocols 88 for transmitting and receiving data wirelessly to and from other components and devices.

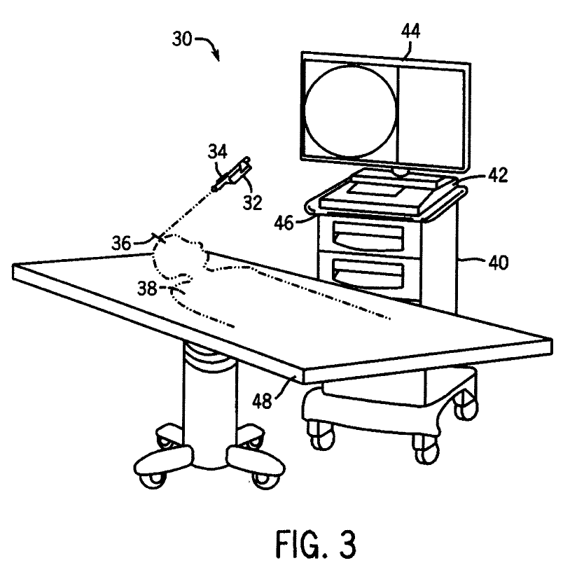

FIG. 3 illustrates a schematic diagram of an exemplary embodiment of a surgical navigation system 30. The surgical navigation system 30 includes at least one magnetoresistance sensor 32 attached to the at least one device 34, and at least one magnetoresistance reference sensor 36 rigidly attached to an anatomical reference of a patient 38 undergoing a medical procedure, and a portable workstation 40. The at least one magnetoresistance reference sensor 36 may also be referred to as a dynamic reference because it is rigidly attached to an anatomical reference of the patient 38 moves along with the patient 38. The portable workstation 40 includes a computer 42, at least one display 44, and a navigation interface 46. The surgical navigation system 30 is configured to operate with the at least one magnetoresistance sensor 32 and the at least one magnetoresistance reference sensor 36 to determine the position and orientation of the at least one device 34. A table 48 is positioned near the portable workstation 40 to support the patient 38 during the medical procedure.

The at least one magnetoresistance sensor 32 may be used to determine one dimension or multiple dimensions of position and/or orientation information (x, y, z, roll, pitch, yaw) relative to the at least one magnetoresistance reference sensor 34, or relative to one or more other magnetoresistance sensors.

The at least one magnetoresistance sensor 32 and at least one magnetoresistance reference sensor 36 are coupled to the navigation interface 46. The at least one magnetoresistance sensor 32 and the at least one magnetoresistance reference sensor 36 are coupled to and communicate to the navigation interface 46 using wireless communication interfaces and protocols. The navigation interface 46 is coupled to the computer 42.

The at least one magnetoresistance reference sensor 36 communicates with and receives data from the at least one magnetoresistance sensor 32. The navigation interface 46 is coupled to and receives data from the at least one magnetoresistance reference sensor 36 and the at least one of magnetoresistance sensor 32. The surgical navigation system 30 provides the ability to track and display the position and orientation of multiple devices 34 having magnetoresistance sensors 32 attached thereto.

In an exemplary embodiment, the at least one magnetoresistance sensor 32 may be configured as battery-powered wireless transmitter or a passive transmitter, and the at least one magnetoresistance reference sensor 36 may be configured as a battery-powered wireless receiver or a passive receiver. It should, however, be appreciated that according to alternate embodiments, the at least one magnetoresistance sensor 32 may be configured as a battery-powered wireless receiver or a passive receiver, and the at least one magnetoresistance reference sensor 36 may be configured as a battery-powered wireless transmitter or a passive transmitter.

In an exemplary embodiment, the at least one magnetoresistance reference sensor 36 generates at least one magnetic field that is detected by at least one magnetoresistance sensor 32. In an exemplary embodiment, the at least one magnetoresistance sensor 32 generates at least one magnetic field that is detected by at least one magnetoresistance reference sensor 36.

The magnetic field measurements may be used to calculate the position and orientation of the at least one device 34 according to any suitable method or system. After the magnetic field measurements are digitized using electronics coupled to the at least one magnetoresistance sensor 32, the digitized signals are transmitted from the at least one magnetoresistance sensor 32 to the navigation interface 46. The digitized signals may be transmitted from the at least one magnetoresistance sensor 32 to the navigation interface 46 using wireless communication interfaces and protocols. The digitized signals received by the navigation interface 46 represent magnetic field information detected by the at least one magnetoresistance sensor 32.

In an exemplary embodiment, the digitized signals received by the navigation interface 46 represent magnetic field information from the at least one magnetoresistance reference sensor 34 detected by the at least one or at least one magnetoresistance sensor 32. The navigation interface 46 transfers the digitized signals to the computer 42. The computer 42 calculates position and orientation information of the at least one device 34 based on the received digitized signals. The position and orientation information may be transmitted from the computer 42 to the display 44 for review by a medical practitioner.

In an exemplary embodiment, the at least one magnetoresistance sensor 32 and the at least one magnetoresistance reference sensor 36 may be powered by a small battery or through inductive coupling.

In an exemplary embodiment, the at least one magnetoresistance sensor 32 may include power conversion and drive circuitry for energizing the sensor. By way of example, a drive current may be supplied by the drive circuitry to energize the sensor, and thereby generate a magnetic field to be detected by the at least one magnetoresistance reference sensor 36. In an exemplary embodiment, the at least one magnetoresistance reference sensor 36 may include power conversion and drive circuitry for energizing the sensor. By way of example, a drive current may be supplied by the drive circuitry to energize the sensor, and thereby generate a magnetic field to be detected by the at least one magnetoresistance sensor 32.

In an exemplary embodiment, the at least one magnetoresistance sensor 32 and the at least one magnetoresistance reference sensor 36 may include storage and processing circuitry for storing and processing data.

In an exemplary embodiment, the at least one magnetoresistance sensor 32 and the at least one magnetoresistance reference sensor 36 may include bi-directional wireless communication circuitry and protocols for transmitting and receiving data.

The surgical navigation system 30 described herein is capable of tracking many different types of devices during different procedures. Depending on the procedure, the at least one device 34 may be a surgical instrument (e.g., an imaging catheter, a diagnostic catheter, a therapeutic catheter, a guidewire, a debrider, an aspirator, a handle, a guide, etc.), a surgical implant (e.g., an artificial disk, a bone screw, a shunt, a pedicle screw, a plate, an intramedullary rod, etc.), or some other device. Depending on the context of the usage of the surgical navigation system 30, any number of suitable devices may be used. In an exemplary embodiment, there may be more than one device 34, and more than one magnetoresistance sensor 32 attached to each device 34.

An exemplary system for implementing the computer 42 may include a general purpose computing device including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules and other data for the computer.

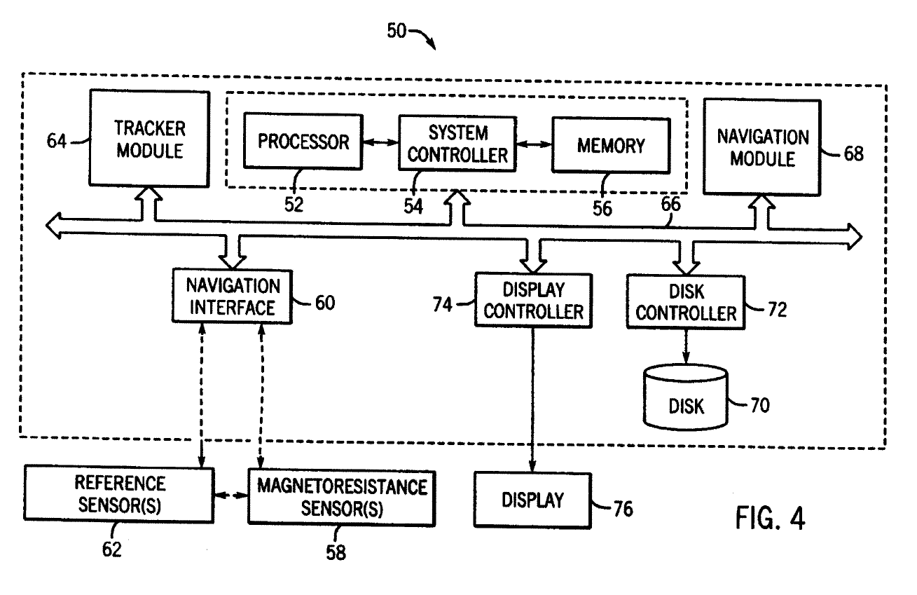

FIG. 4 illustrates a block diagram of an exemplary embodiment of a surgical navigation system 50. The surgical navigation system 50 is illustrated conceptually as a collection of modules, but may be implemented using any combination of dedicated hardware boards, digital signal processors, field programmable gate arrays, and processors. Alternatively, the modules may be implemented using an off-the-shelf computer with a single processor or multiple processors, with the functional operations distributed between the processors. As an example, it may be desirable to have a dedicated processor for position and orientation calculations as well as a dedicated processor for visualization operations. As a further option, the modules may be implemented using a hybrid configuration in which certain modular functions are performed using dedicated hardware, while the remaining modular functions are performed using an off-the-shelf computer. In the embodiment shown in FIG. 4, the system 50 includes a processor 52, a system controller 54 and memory 56. The operations of the modules may be controlled by the system controller 54.

At least one magnetoresistance sensor 58 and at least one magnetoresistance reference sensor 62 are wirelessly coupled to a navigation interface 60. The surgical navigation system 50 may be configured to assign a unique identifier to each magnetoresistance sensor 58 and each magnetoresistance reference sensor 62 through the navigation interface 60, so that the surgical navigation system 50 can identify which magnetoresistance sensor is attached to which device, or which magnetoresistance reference sensor 62 is attached to which anatomical reference. In an exemplary embodiment, the at least one magnetoresistance sensor 58 generates at least one magnetic field that is detected by the at least one magnetoresistance reference sensor 62. In an exemplary embodiment, the at least one magnetoresistance reference sensor 62 generates at least one magnetic field that is detected by the at least one magnetoresistance sensor 58.

In an exemplary embodiment, the at least one magnetoresistance sensor 58 may be configured as a transmitter or magnetic field generator, and the at least one magnetoresistance reference sensor 62 may be configured as a magnetic field receiver. It should, however, be appreciated that according to alternate embodiments the at least one magnetoresistance sensor 58 may be configured as a magnetic field receiver, and the at least one magnetoresistance reference sensor 62 may be configured as a magnetic field generator.

The navigation interface 60 receives and/or transmits digitized signals from the at least one magnetoresistance sensor 58 or the at least one magnetoresistance reference sensor 62. The navigation interface 60 may include at least one Ethernet port. The at least one port may be provided, for example, with an Ethernet network interface card or adapter. However, according to various alternate embodiments, the digitized signals may be transmitted from the at least one magnetoresistance sensor 58 or the at least one magnetoresistance reference sensor 62 to the navigation interface 60 using wireless communication interfaces and protocols.

The digitized signals received by the navigation interface 60 represent magnetic field information from the at least one magnetoresistance sensor 58 detected by the at least one magnetoresistance reference sensor 62. In an alternative embodiment, the digitized signals received by the navigation interface 60 represent magnetic field information from the at least one magnetoresistance reference sensor 62 detected by the at least one magnetoresistance sensor 58. The navigation interface 60 transmits the digitized signals to a tracker module 64 over a local interface 66. In an exemplary embodiment, the local interface 66 is a peripheral component interconnect (PCI) bus. However, according to various alternate embodiments, equivalent bus technologies may be substituted. In an exemplary embodiment, the tracker module 64 calculates position and orientation information based on the received digitized signals. This position and orientation information provides a location of a device. The tracker module 64 communicates the position and orientation information to a navigation module 68 over the local interface 66.

Upon receiving the position and orientation information, the navigation module 68 is used to register the location of the device to acquired patient data. In the embodiment illustrated in FIG. 4, the acquired patient data is stored on a disk 70. The acquired patient data may include computed tomography (CT) data, magnetic resonance (MR) data, positron emission tomography (PET) data, ultrasound data, x-ray data, or any other suitable data, as well as any combinations thereof By way of example only, the disk 70 is a hard disk drive, but other suitable storage devices may be used.

The acquired patient data is loaded into memory 56 from the disk 70. The acquired patient data is retrieved from the disk 70 by a disk controller 72. The navigation module 68 reads from memory 56 the acquired patient data. The navigation module 68 registers the location of the device to acquired patient data, and generates image data suitable to visualize the patient image data and a representation of the device. In the embodiment illustrated in FIG. 4, the image data is transmitted to a display controller 74 over the local interface 66. The display controller 74 is used to output the image data to a display 76.

Various display configurations may be used to improve operating room ergonomics, display different views, or display information to personnel at various locations. For example, as illustrated in FIG. 3, at least one display 44 may be included with the surgical navigation system 30. The at least one display 44 may include two or more separate displays or a large display that may be partitioned into two or more display areas. Alternatively, the at least one display 44 may be mounted on a surgical boom extending from a ceiling or wall of an operating room. The surgical boom may be mounted to and extend from a ceiling or wall of an operating room, attachable to a surgical table, or mounted on a portable cart.

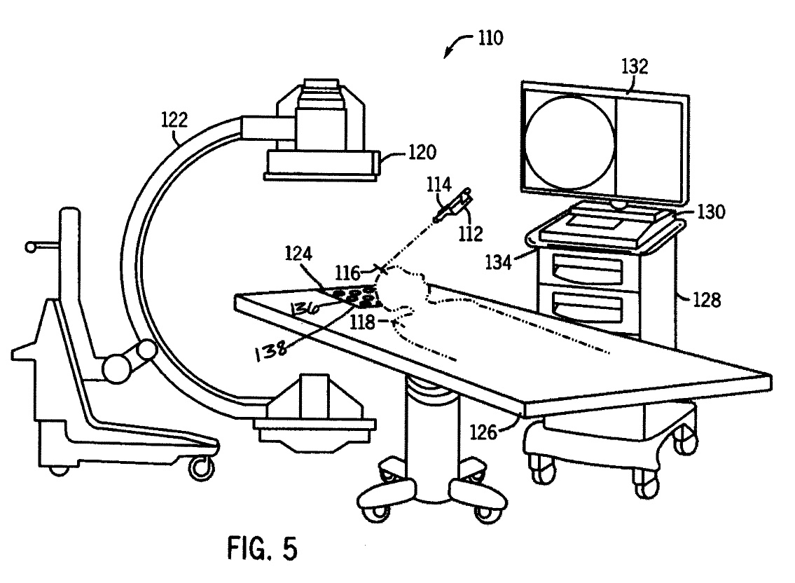

FIG. 5 illustrates a schematic diagram of an exemplary embodiment of a surgical navigation system 110. The surgical navigation system 110 includes at least one magnetoresistance sensor 112 attached to at least one device 114, a first magnetoresistance reference sensor 116 rigidly attached to an anatomical reference of a patient 118 undergoing a medical procedure, a second magnetoresistance reference sensor 120 attached to an imaging apparatus 122, a planar sensor array 124 positioned on a table 126 supporting the patient 118, and a portable workstation 128. In an exemplary embodiment, the imaging apparatus 122 is a mobile fluoroscopic imaging apparatus. The portable workstation 128 includes a computer 130, at least one display 132, and a navigation interface 134. The surgical navigation system 110 is configured to operate with the at least one magnetoresistance sensor 112, the first and second magnetoresistance reference sensors 116, 120, and the planar sensor array 124 to determine the position and orientation of the at least one device 114.

The at least one magnetoresistance sensor 112, the first and second magnetoresistance reference sensors 116, 120, and the planar sensor array 124 are coupled to the navigation interface 134. The at least one magnetoresistance sensor 112, the first and second magnetoresistance reference sensors 116, 120, and the planar sensor array 124 are coupled to and communicate to the navigation interface 134 through a wireless connection. The navigation interface is coupled to the computer 130.

The at least one magnetoresistance sensor 112 communicates with and transmits/receives data from the first and second magnetoresistance reference sensors 116, 120, and the planar sensor array 124. The navigation interface 134 is coupled to and receives data from the at least one magnetoresistance sensor 112 communicates with and transmits/receives data from the first and second magnetoresistance reference sensors 116, 120, and the planar sensor array 124. The surgical navigation system 110 provides the ability to track and display the position and orientation of multiple devices 114 having magnetoresistance sensors 112 attached thereto. The position and orientation information may be transmitted from the computer 130 to the display 132 for review by a medical practitioner.

In an exemplary embodiment, the planar sensor array 124 includes a plurality of sensors 136 formed on or within a substrate 138. The plurality of sensors 136 may be made of a conductive material. The substrate 138 may be made of an insulating material that is rigid or flexible. In an exemplary embodiment, the planar sensor array 124 may be incorporated into the table 126, a tablemat, or a table draping.

In an exemplary embodiment, the at least one magnetoresistance sensor 112 and the first and second magnetoresistance reference sensors 116, 120, and the planar sensor array 124 may be configured as transmitters or magnetic field generators, or configured as magnetic field receivers, depending on the application.

In an exemplary embodiment, the planar sensor array 124 may be a planar transmitter coil array that includes a plurality of transmitter coils 136 formed on or within a substrate 138. The plurality of transmitter coils 136 may be made of a conductive material. The substrate 138 may be made of an insulating material that is rigid or flexible. In an exemplary embodiment, the planar transmitter coil array may be incorporated into the table 126, a tablemat, or a table draping.

In an exemplary embodiment, the at least one magnetoresistance sensor 112 and the first and second magnetoresistance reference sensors 116, 120 may be powered by a small battery or through inductive coupling.

In an exemplary embodiment, the at least one magnetoresistance sensor 112 and the first and second magnetoresistance reference sensors 116, 120 may include power conversion and drive circuitry for energizing the sensors.

In an exemplary embodiment, the at least one magnetoresistance sensor 112 and the first and second magnetoresistance reference sensors 116, 120 may include storage and processing circuitry for storing and processing data.

In an exemplary embodiment, the at least one magnetoresistance sensor 112 and the first and second magnetoresistance reference sensors 116, 120, and the planar sensor array 124 may include bi-directional wireless communication circuitry and protocols for transmitting and receiving data.

The surgical navigation system 110 described herein is capable of tracking many different types of devices during different procedures. Depending on the procedure, the at least one device 114 may be a surgical instrument (e.g., an imaging catheter, a diagnostic catheter, a therapeutic catheter, a guidewire, a debrider, an aspirator, a handle, a guide, etc.), a surgical implant (e.g., an artificial disk, a bone screw, a shunt, a pedicle screw, a plate, an intramedullary rod, etc.), or some other device. Depending on the context of the usage of the surgical navigation system 110, any number of suitable devices may be used. In an exemplary embodiment, there may be more than one device 114, and more than one magnetoresistance sensor 112 attached to each device 114.

In an exemplary embodiment, a magnetoresistance reference sensor is fixed to an anatomical reference, a first magnetoresistance sensor is fixed to a first device or implant, and a second magnetoresistance sensor is fixed to a second device, implant or imaging apparatus.

In an exemplary embodiment, a planar sensor array is positioned on a surgical table, a magnetoresistance reference sensor is fixed to an anatomical reference, and a plurality of magnetoresistance sensors are fixed to devices, implants, patient body parts, and/or an imaging device.

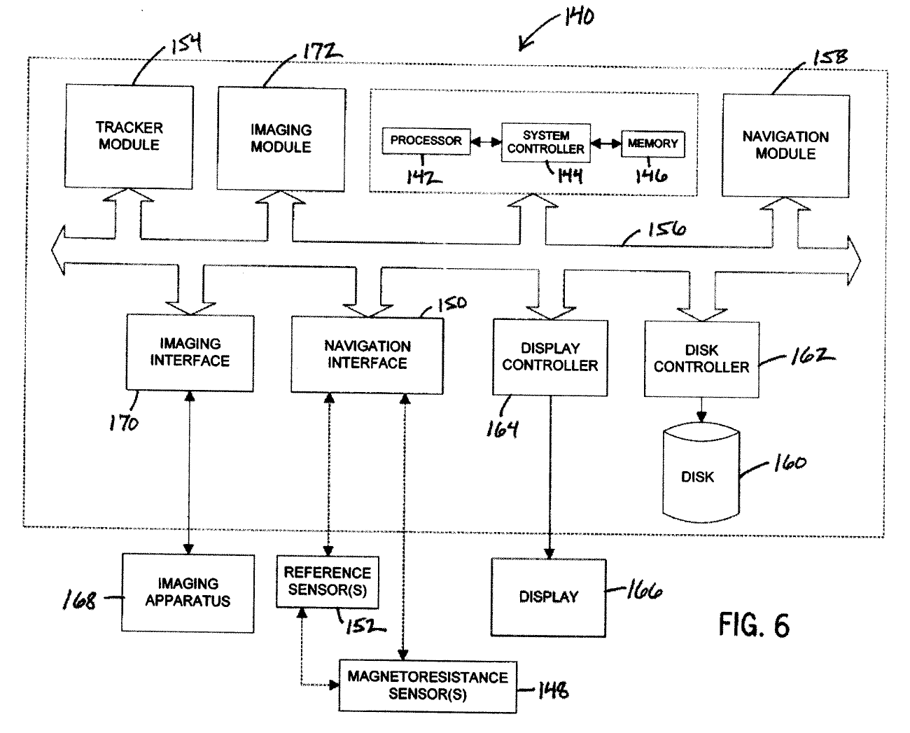

FIG. 6 illustrates a block diagram of an exemplary embodiment of a surgical navigation system 140. The surgical navigation system 140 is illustrated conceptually as a collection of modules, but may be implemented using any combination of dedicated hardware boards, digital signal processors, field programmable gate arrays, and processors. Alternatively, the modules may be implemented using an off-the-shelf computer with a single processor or multiple processors, with the functional operations distributed between the processors. As an example, it may be desirable to have a dedicated processor for position and orientation calculations as well as a dedicated processor for visualization operations. As a further option, the modules may be implemented using a hybrid configuration in which certain modular functions are performed using dedicated hardware, while the remaining modular functions are performed using an off-the-shelf computer. In the embodiment shown in FIG. 6, the system 140 includes a processor 142, a system controller 144 and memory 146. The operations of the modules may be controlled by the system controller 144.

At least one magnetoresistance sensor 148 and at least one magnetoresistance reference sensor 152 are wirelessly coupled to a navigation interface 150. An imaging apparatus 168 is coupled to an imaging interface 170.

In an exemplary embodiment, the at least one magnetoresistance sensor 148 generates at least one magnetic field that is detected by the at least one magnetoresistance reference sensor 152. In an exemplary embodiment, the at least one magnetoresistance reference sensor 152 generates at least one magnetic field that is detected by the at least one magnetoresistance sensor 148.

In an exemplary embodiment, the at least one magnetoresistance sensor 148 may be configured as a transmitter or magnetic field generator, and the at least one magnetoresistance reference sensor 152 may be configured as a magnetic field receiver. It should, however, be appreciated that according to alternate embodiments the at least one magnetoresistance sensor 148 may be configured as a magnetic field receiver, and the at least one magnetoresistance reference sensor 152 may be configured as a transmitter or magnetic field generator.

The navigation interface 150 receives and/or transmits digitized signals from the at least one magnetoresistance sensor 148 or the at least one magnetoresistance reference sensor 152. The digitized signals may be transmitted from the at least one magnetoresistance sensor 148 or the at least one magnetoresistance reference sensor 152 to the navigation interface 150 using wireless communication interfaces and protocols.

The digitized signals received by the navigation interface 150 represent magnetic field information from the at least one magnetoresistance sensor 148 detected by the at least one magnetoresistance reference sensor 152. In an alternative embodiment, the digitized signals received by the navigation interface 150 represent magnetic field information from the at least one magnetoresistance reference sensor 152 detected by the at least one magnetoresistance sensor 148. The navigation interface 150 transmits the digitized signals to a tracker module 154 over a local interface 156. In an exemplary embodiment, the local interface 156 is a PCI bus. In an exemplary embodiment, the tracker module 154 calculates position and orientation information based on the received digitized signals. This position and orientation information provides a location of a device. The tracker module 154 communicates the position and orientation information to a navigation module 158 over the local interface 156.

Upon receiving the position and orientation information, the navigation module 158 is used to register the location of the device to acquired patient data. The patient data may be acquired by the imaging apparatus 168, transmitted to the imaging interface 170, which is coupled to the local interface 156. The acquired patient data may be processed by an imaging module 172 that is coupled to the local interface 156. The acquired patient data may be stored on a disk 160. The acquired patient data may include real-time fluoroscopic imaging data from imaging apparatus 168 or previously acquired CT data, MR data, PET data, ultrasound data, x-ray data, or any other suitable data, as well as any combinations thereof. By way of example only, the disk 160 is a hard disk drive, but other suitable storage devices may be used.

The acquired patient data is loaded into memory 146 from the disk 160. The acquired patient data is retrieved from the disk 160 by a disk controller 162. The navigation module 158 reads from memory 146 the acquired patient data. The navigation module 158 registers the location of the device to acquired patient data, and generates image data suitable to visualize the patient image data and a representation of the device. The image data is transmitted to a display controller 164 over the local interface 156. The display controller 164 is used to output the image data to a display 166.

Certain embodiments may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers (PCs), hand-held devices, multi-processor systems, microprocessor-based or programmable electronics, network PCs, minicomputers, mainframe computers, and the like. Embodiments of the disclosure may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

While the disclosure has been described with reference to various embodiments, those skilled in the art will appreciate that certain substitutions, alterations and omissions may be made to the embodiments without departing from the spirit of the disclosure. Accordingly, the foregoing description is meant to be exemplary only, and should not limit the scope of the disclosure as set forth in the following claims.

What is claimed is:

1. A surgical navigation system comprising:
at least one wireless magnetoresistance reference sensor rigidly attached to an anatomical reference of a patient;
at least one wireless magnetoresistance sensor attached to at least one device; and
at least one processor for determining the position and orientation of the at least one device.

2. The surgical navigation system of claim 1, wherein the at least one wireless magnetoresistance reference sensor comprises an insulating substrate, an alternating pattern of a metal material and a semiconductor material deposited on a surface of the insulating substrate, and a bias magnet material deposited over the alternating pattern of the metal material and the semiconductor material.

3. The surgical navigation system of claim 1, wherein the at least one wireless magnetoresistance sensor comprises an insulating substrate, an alternating pattern of a metal material and a semiconductor material deposited on a surface of the insulating substrate, and a bias magnet material deposited over the alternating pattern of the metal material and the semiconductor material.

4. The surgical navigation system of claim 3, wherein the bias magnet material subjects the semiconductor material to a magnetic field.

5. The surgical navigation system of claim 4, wherein the at least one wireless magnetoresistance sensor provides a signal in response to a strength and a direction of the magnetic field.

6. The surgical navigation system of claim 1, wherein the at least one wireless magnetoresistance sensor has an active area of approximately 0.1 mm by 0.1 mm in size.

7. The surgical navigation system of claim 1, wherein the at least one wireless magnetoresistance reference sensor and the at least one wireless magnetoresistance reference sensor are powered by a small battery.

8. The surgical navigation system of claim 1, wherein the at least one wireless magnetoresistance reference sensor and the at least one wireless magnetoresistance sensor are powered through inductive coupling.

9. The surgical navigation system of claim 1, wherein the at least one wireless magnetoresistance reference sensor is a passive transmitter and the at least one wireless magnetoresistance sensor is a passive receiver.

10. The surgical navigation system of claim 1, wherein the at least one wireless magnetoresistance reference sensor is a passive receiver and the at least one wireless magnetoresistance sensor is a passive transmitter.

11. The surgical navigation system of claim 1, further comprising bi-directional wireless communication circuitry coupled to the at least one wireless magnetoresistance reference sensor and the at least one wireless magnetoresistance sensor for transmitting and receiving data wirelessly.

12. A surgical navigation system comprising:
at least one wireless magnetoresistance sensor attached to at least one device;
a planar sensor array positioned on a table supporting a patient undergoing a medical procedure; and
at least one processor for determining the position and orientation of the at least one device.

13. The surgical navigation system of claim 12, further comprising a wireless magnetoresistance reference sensor attached to an anatomical reference of the patient.

14. The surgical navigation system of claim 12, further comprising an imaging apparatus.

15. The surgical navigation system of claim 14, further comprising a wireless magnetoresistance reference sensor attached to the imaging apparatus.

16. The surgical navigation system of claim 12, wherein the planar sensor array includes a plurality of sensors formed on a substrate.

17. The surgical navigation system of claim 12, wherein the planar sensor array is a transmitter coil array that includes a plurality of transmitter coils formed on a substrate.

18. The surgical navigation system of claim 12, further comprising bi-directional wireless communication circuitry coupled to the at least one wireless magnetoresistance sensor and the at least one planar sensor array for transmitting and receiving data wirelessly.

19. An integrated circuit device for use in a surgical navigation system comprising:
a magnetoresistance sensor; and
additional circuitry coupled to the magnetoresistance sensor;
wherein the magnetoresistance sensor and the additional circuitry coupled to the magnetoresistance sensor operate wirelessly.

20. The integrated circuit device of claim 19, wherein the magnetoresistance sensor comprises an insulating substrate, an alternating pattern of a metal material and a semiconductor material deposited on a surface of the insulating substrate, and a bias magnet material deposited over the alternating pattern of the metal material and the semiconductor material.

21. The integrated circuit device of claim 20, wherein the bias magnet material subjects the semiconductor material to a magnetic field.

22. The integrated circuit device of claim 21, wherein the magnetoresistance sensor provides a signal in response to a strength and a direction of the magnetic field.

23. The integrated circuit device of claim 19, wherein the magnetoresistance sensor has an active area of approximately 0.1 mm by 0.1 mm in size.

24. The integrated circuit device of claim 19, wherein the magnetoresistance sensor is powered by a small battery or through inductive coupling.

25. The integrated circuit device of claim 19, wherein the magnetoresistance sensor is attached to at least one of a surgical instrument, a surgical implant, or other medical device.

26. The integrated circuit device of claim 19, wherein the magnetoresistance sensor is attached to at least one of tissue, a bone, or an organ of a patient.

27. The integrated circuit device of claim 19, wherein the magnetoresistance sensor is a passive transmitter.

28. The integrated circuit device of claim 19, wherein the magnetoresistance sensor is a passive receiver.

29. The integrated circuit device of claim 19, wherein the additional circuitry includes power conversion and drive circuitry coupled to the magnetoresistance sensor for energizing the magnetoresistance sensor.

30. The integrated circuit device of claim 19, wherein the additional circuitry includes storage and processing circuitry coupled to the magnetoresistance sensor for storing and processing data from and to the magnetoresistance sensor.

31. The integrated circuit device of claim 19, wherein the additional circuitry includes bi-directional wireless communication circuitry coupled to the magnetoresistance sensor for transmitting and receiving data wirelessly from and to the magnetoresistance sensor.
